(19) United States
(12) Patent Application Publication
(10) Pub. No.: US 2005/0152887 A1
(43) Pub. Date: Jul. 14, 2005

(54) Protonic formulation

(75) Inventor: Michael Ernest, Chula Vista, CA (US)

Correspondence Address: FRANK G MORKUNAS, 7750 DAGGET ST, SUITE 203, SAN DIEGO, CA, 92111, US

(73) Assignee: Doctor's Signature Sales and Marketing International Corp. [DBA Life Force International

(21) Appl. No.: 10/757,706

(22) Filed: Jan. 14, 2004

(51) Int. Cl. A61K 38/54

(52) U.S. Cl. 424/94.2

(57) ABSTRACT

A stabilized protonic mixture having a protonic formulation (FIG. 2A) comprised primarily of proteins (FIG. 2A), enzymes and pH adjusters (FIG. 2A), all in specific ratios to one another; a liquid medium which, when combined to the protonic formulation, initiates activation of the amino acids within the protonic formulation; and a stabilizing component which stabilizes the amino acids during the process of their activation.

### CROSS REFERENCES AND RELATED APPLICATIONS

This application is the National Stage of International Application No. PCT/US 02/24,662, filed on Aug. 2, 2002, which claims the benefit under 35 U.S.C. § 19(e) of U.S. Provisional Application No. 60/311,280 filed on Aug. 9, 2001.

### STATEMENT REGARDING FEDERALLY-SPONSORED RESEARCH OR DEVELOPMENT

None

### BACKGROUND OF THE INVENTION

This present invention relates to an improvement in protein formulations, and more particularly to a formulation and mixture which unprotonates and stabilizes unprotonated amino acids for utilization within the body of greater concentrations of the dissociated amino acids.

A major component of most food groups is protein from which dietary energy and the basic ‘building blocks’ of and for the body are derived. The proteins ingested by the body are not the same proteins required by the body nor, since they comprise large molecules, can they properly be absorbed and utilized without alteration by digestion. When protein is ingested, it must first be digested to form the component amino acids needed by the body. These amino acids, when activated, are absorbed into the blood stream and/or are used to form other necessary protein-based molecules needed by the body. Amino acids are used in the body in several ways. The most common of which are protein synthesis (to produce the necessary protein-based molecules needed to ‘build’ and/or ‘repair’ and/or ‘maintain’ various body parts such as muscle tissue, and production of anti-bodies to promote healing or resistence), synthesis of other compounds (such as, but not limited to, components of nucleotides, catecholamines, neurotransmitters, histamine, and porphyrins, to name a few), and as a biological fuel and energy producer.

During the digestion phase, enzymes react with the protein to breakdown large protein molecules into smaller molecules of charged or ionized amino acids. The enzymatic digestive process begins in the mouth during chewing to break down larger bites of food, secretion of saliva to moisten the foods and thereby aid in the chewing and break-down process, and secretion of alpha-amylase which initiates starch digestion by breaking down complex carbohydrates into sugars. The process continues in the stomach which is highly acidic. This acidity aids in destroying non-beneficial ingested bacteria, activates further enzymatic activity there, and initiates further protein digestion and activation process. The digestive process continues further at the small intestine where activated amino acids are more readily absorbed into the blood stream and utilized in the reconstructive process of building, repairing, or maintaining as needed. Though most of the digestive process is completed at and within the small intestine, the large intestine completes the process if needed.

Proteins are a diverse group of biological molecules over which it is believed that several billions of protein groups are extant in nature. Regardless of such diversity, all protein groups generally share the same basic structure in that all are chains of sub-units known as amino acids of which there are 300 different amino acids extant in nature and only 20 occurring in natural biological proteins—these 20 amino acids are present in all known forms of life. When these different amino acids are arranged in different combinations they make up all the different protein groups.

The basic amino acid structure in proteins comprises a variable side chain (“R”), a carbon atom (“C”), an amino or nitrogen group (“NH2”), and a carboxyl group (“COOH”). Simply put, the primary structure of a protein is its amino acid sequence formed when a peptide bond joins the carboxyl group of one amino acid to the amino group of another amino acid. A long chain forms from many amino acids with one molecule of water being released with the formation of each peptide link. The amino groups and the carboxyl groups comprise the terminal ends which become, or should become, activated (unprotonated) during the digestive process and, only when activated, become utilizable. If the amino acid does not become activated, it will not be utilized but ultimately will be eliminated from the body. The more amino acids activated, the greater utilization for repair, maintenance, and generation as needed.

Generally speaking, enzymes ready ingested proteins into amino acids for activation. Once the amino acids are activated, if they are not utilized, they are eliminated. Pre-activated amino acids are in what is referred to as a protonated form, when activated they are in what is referred to as an unprotonated form. Carboxyl groups and amino groups are of biological functional groups of weak acids or weak bases. The dissociation behavior (i.e, activation), or protonic equilibria, of carboxyl groups and amino groups is based on their relative reaction to the intracellular pH levels to which exposed. In short, each carboxyl group and each amino group for a specific amino acid will remain in protonated form, or return to protonated form, based on the pH at which it dissociates (i.e., displaces or ionizes a hydrogen atom) and becomes unprotonated or activated (the protonic state). As the pH phase shifts upward and downward so too do the various phases of protonation/unprotonation of amino acids until they attain the respective pK level or are eliminated from the body.

Generally, the protonated form of an acid is also referred to as ‘the acid’ and the unprotonated form is referred to as the ‘conjugate base’ of ‘the acid’ (reference may also be reversed; i.e., a ‘base’ and its ‘conjugate acid’ where applicable). The relative strengths of such weak acids and weak bases are expressed quantitatively as their dissociation constants which relates to their respective tendencies to ionize. The dissociation constant is expressed by the letter “K” and, since the numerical values of K for weak acids and weak bases are negative exponential numbers, they are expressed in the following manner: “pK”. The pK is related to K as pH is to H⁺ concentration. Therefore, when the associated (protonated or non-activated state) and dissociated (unprotonated or conjugate base or activated state) species are present in equal concentration, the prevailing hydrogen ion concentration (H⁺) is numerically equal to the dissociation constant (K or pK). Expressed in pK terms, the pK of an acid group is that pH at which the protonated and unprotonated species or forms are present at equal concentrations.

Amino acids have at least two ionizable weak acid groups; the carboxyl group (‘—COOH’) and the amino group (‘—NH₃’). In solution, generally only one of these two groups is charged (activated) and one is uncharged (non-activated). Where ‘R’ is the variable group side chain attached to the central carbon atom, ‘R—COOH’ and ‘R—NH₃’ represent the protonated or acidic couples in the equilibria, and ‘R—COO⁻’ AND ‘R—NH₂’ represent the conjugate bases (also referred to a proton acceptor) of the respective corresponding acids. As the pH levels in the body change, the pK of different amino acids is achieved, the amino acids become activated and readily utilizable. The pH level and, concomitantly, the pK may fluctuate back and forth, attaining and passing the protonic equilibria, attaining and losing the conjugate base and activated state, until some of the activated amino acids are utilized while the greater majority are eliminated, not utilized and, thereby, wasted.

One's ability to react to the various pH phase shifts caused during the digestive process and caused by various enzymes is affected by that person's age, health, dietary habits, and stresses of life. One's ability to produce respective digestive enzymes efficiently and in sufficient quantity diminishes thereby decreasing the amounts of unprotonated amino acids within the digestive system and, concomitantly, decreasing the amounts utilized for building, repairing, and maintaining. In order to establish and maintain a protonic solution (unprotonated form) of amino acids, several conditions must be present. The pH and digestive substances must be readily available, there must be in sufficient quantities therefor, and there should be a stabilizing component to maintain the amino acids in their unprotonated form. Protein is readily available in supplements and in various food products. Activating enzymes are available also in supplements and in the natural digestive process. What is not readily available is the unique formulation of the present invention; and a stabilizing component to maintain an unprotonated state, or within utilizable limits of an unprotonated state, of amino acids when in or near that state.

If protein could be activated before ingestion and stabilized in, or within a range of, its most unprotonated form before ingestion, more bio-available (i.e., utilizable) amino acids would be present in the body for utilization. Such is the purpose of the present invention.

Accordingly, several objects and advantages of my invention are to:
a. provide to a user utilizable amino acids (unprotonated state) prior to normal ingestion;
b. stabilize unprotonated amino acids such that greater concentrations may be utilized;
c. aid in the natural digestive process by stabilizing unprotonated amino acids which are activated by the natural digestive process thereby increasing the concentration of unprotonated amino acids available for utilization;
d. provide a formulation which accomplishes all the above in an easy-to-prepare and easy-to-use manner.

The foregoing has outlined some of the more pertinent objects of the present invention. These objects should be construed to be merely illustrative of some of the more prominent features and applications of the intended invention. Many other beneficial results can be attained by applying the disclosed invention in a different manner or by modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### BRIEF SUMMARY OF THE INVENTION

The above-noted problems, among others, are overcome by the present invention. Briefly stated, the present invention contemplates a stabilized protonic mixture having a protonic formulation [PF] comprised primarily of proteins [PM], enzymes and pH adjusters [EAF], all in specific ratios to one another; a liquid medium which, when combined to the protonic formulation, initiates activation of the amino acids within the protonic formulation; and a stabilizing component which stabilizes the amino acids during the process of their activation.

The foregoing has outlined the more pertinent and important features of the present invention in order that the detailed description of the invention that follows may be better understood so the present contributions to the art may be more fully appreciated. Additional features of the present invention will be described hereinafter which form the subject of the claims. It should be appreciated by those skilled in the art that the conception and the disclosed specific embodiment may be readily utilized as a basis for modifying or designing other structures and methods for carrying out the same purposes of the present invention. It also should be realized by those skilled in the art that such equivalent constructions and methods do not depart from the spirit and scope of the inventions as set forth in the appended claims.

### BRIEF DESCRIPTION OF THE DRAWINGS

For a fuller understanding of the nature and objects of the invention, reference should be had to the following detailed description taken in conjunction with the accompanying drawings in which:

FIG. 1 is a schematic view of dissociation and utilization of amino acids during the normal digestive process.

FIGS. 2A and 2B are schematic views of dissociation and utilization of amino acids during the protonic digestive process

### DETAILED DESCRIPTION OF THE INVENTION

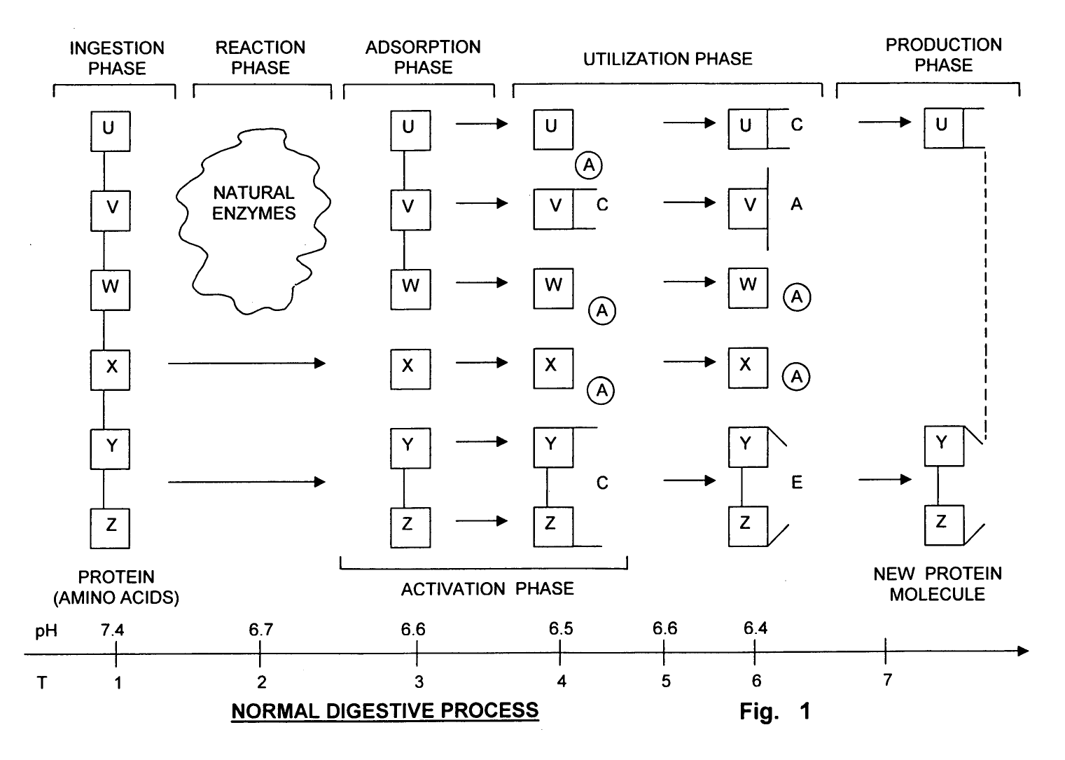

Referring now to the drawings for reference, FIG. 1 schematically illustrates the normal digestive process. The purpose of the figures is not to prove a hypothesis, but to visually demonstrate its concept and to aid in its understanding. In this regard, the square blocks containing reference characters ‘u’ through ‘z’ represent a protein source to be broken down into various protein molecules and their respective amino acids if conditions warrant. The time line (‘T’) at the bottom of the figure represents a time sequence and is not representative of time units (such as seconds, minutes, hours, days, and the like). The pH values for each time line or time column are for illustrative purposes to assist in the visualization of an amino acid becoming activated or remaining non-activated or at various stages in between. Reference letters ‘A’ through ‘E’ represent various stages of the activation cycle, which, based on the pK for the specific amino acid and the current pH, may cycle from A to C and back to A or B; or may cycle from A to C to E for example. For illustration purposes only, A=5% activation, B=25% activation, C=100% activation (as near to the ideal pK value for that specific amino acid), D=25% activation after having attained 100% activation without being utilized at full activation, and E=25% activation also after having attained 100% activation without being utilized at full activation. Reference character A with a circle therearound represents pre-activation.

Time phase 1 is the ingestion of the protein source followed shortly thereafter by the reaction phase of natural enzymes reacting upon the protein source at time phase 2. The activation phase at time lines 3 and 4 includes the adsorption phase and part of the utilization phase. Time lines 4-6 represent the utilization phase resulting ultimately in formation of a new protein molecule at time line 7.

Referring to time line 3, protein source u-v-w-x-y-z has been broken down into and formed one tri-peptide amino acid molecule (u-v-w), one mono-peptide amino acid molecule (x) and one di-peptide amino acid molecule (y-z). These are the basic amino acid building blocks; as a result of continued enzymatic action, di-peptides can be further broken down to mono-peptides and tri-peptides can be further broken into di-peptides and ultimately mono-peptides as is illustrated in FIG. 1 from time line 3 to time line 4 wherein the tri-peptide amino acid molecule (u-v-w) was further broken down into three mono-peptide amino acid molecules (u, v, and w).

During the utilization phase at time line 6, amino acid u has become fully activated and will form part of the final new protein molecule in our example at time line 7 along with amino acid y-z, which passed over its full activation state and is utilized in a partially activated state.

This represents that the pK for amino acid u is about 6.4 whereas the pK for amino acid y-z is higher (less acidic) than 6.4 (see time line 4 at pH 6.6 at where it was in a fully activated state. Amino acids w and x remained in a non-activated state during utilization and are therefore not utilized. Their pK could be at a higher or lower pH level; i.e., passed over it or had not yet attained it. As for amino acid v, from pH 6.5 to 6.4 (time lines 4 to 6) it reverted to A from C, while during the same time line periods amino acid y-z continued past C to E. This example suggests that the pK for amino acid v is slightly higher than the pK for amino acid y-z.

The protonic formulation (referred to as PF) of the present invention contains a protein mixture (referred to as PM and to be described below) and a mixture of enzymes and pH adjusters (for administrative clarity the enzyme-pH adjustor formulation will be referred to as enzyme activator formulation, identified as EAF, and also described below). The specific enzymes and pH adjustors selected for the enzyme activator formulation are important for proper activation, pH adjustment, and attainment of pK for the amino acids and optimization of bio-availability of the amino acids. The optimum ratio of enzyme activator formulation (EAF) to protein mixture is about 1 part EAF to 25 parts of protein mixture, though 1 part EAF to between about 10 to 30 parts protein mixture will function suitably for the intended purpose. The EAF is optimally comprised of:

| | |
|---|---|
| a. Betaine HCl - 4.0% | b. Pepsin - 1.5% |
| c. Trypsin - 0.4% | d. Chymotrypsin - 0.3% |
| e. Protease - 0.4% | f. Bromolein - 0.5% |
| g. Papaya - 0.6% | h. Vitamin C - 5.0% |
| i. Lemon powder - 0.6% | j. Glutamic acid - 0.2% |
| k. Glycine - 86.4% | |

Modifying these ratios (as expressed above by percentage amounts) by 20% up or down will still provide a suitable formulation suited for the intended purpose (i.e., a 20% downward swing in the amount of glycine yields glycine to be about 69.12% of the total EAF content). I have found, however, that the optimum ratios, as expressed above in percentages, cause a beneficial synergistic effect on activation and pH adjustments for the proteins utilized in the protonic formulation (PF).

The protein sources and mixture I have found to work best with the enzyme activator formulation (EAF) above include the following in the following quantities:
a. Whey protein isolate—30.0%
b. Instant whey concentrate—15.0%
c. Soy protein isolate—25.0%
d. Pea protein—5.0%
e. Rice protein—5.0%
f. Maltodextrin—15.7%
g. Steviocide—0.3%
h. French vanilla flavor—1.75% (not a protein source)
i. Peach mango flavor—0.5% (not a protein source)
j. Xanthan gum—0.5%
k. Lecithin—0.5%
l. Tricalcium phosphate—0.75%

The above mixture of substances, primarily protein sources, is referred to as the protein mixture [or PM]. Modifying these ratios (as expressed above by percentage amounts) by 20% up or down will, as with the EAF, still provide a suitable formulation suited for the intended purpose. I have found, however, that the optimum ratios, as expressed above in percentages, create the greatest beneficial synergistic effect on activation and ultimate utilization. A serving size suited for the average person is about 26 grams which, optimally, comprise about 1 gram of the EAF and about 25 grams of the protein mixture.

The carrier for best activation, stabilization, and ultimate utilization is a liquid, preferably water, through any non-toxic liquid medium will suffice. Based on a 26-gram protonic formulation (PF) described above (i.e., EAF and protein mixture), between about 120-240 ml of water will suffice to initiate reaction and activation of the PF. The activation process will initiate the cycles (pre-A through E and back, if warranted by the pH level and pK of the specific amino acids involved) as illustrated, by way of example, in FIG. 1. In the absence of a stabilizing element, many amino acids, as in the normal digestive process, will be spent, become non-utilizable, and will thereby be eliminated without utilization. With use of the present invention, addition of water initiates the protonic activation phase to be followed by stabilization. Stabilization is realized by application of approximately between 2-10 ml of glycerin in any form to the solution described above containing 26 grams of PF and 120-240 ml water. Optimum amount of glycerin, however, is 7.5 ml. This amount of glycerin, added to the 26 grams of PF and 180 ml water will produce the greatest amounts of stabilized amino acids. The solution containing the PF, the liquid medium, and the stabilizing component is referred to as the protonic mixture.

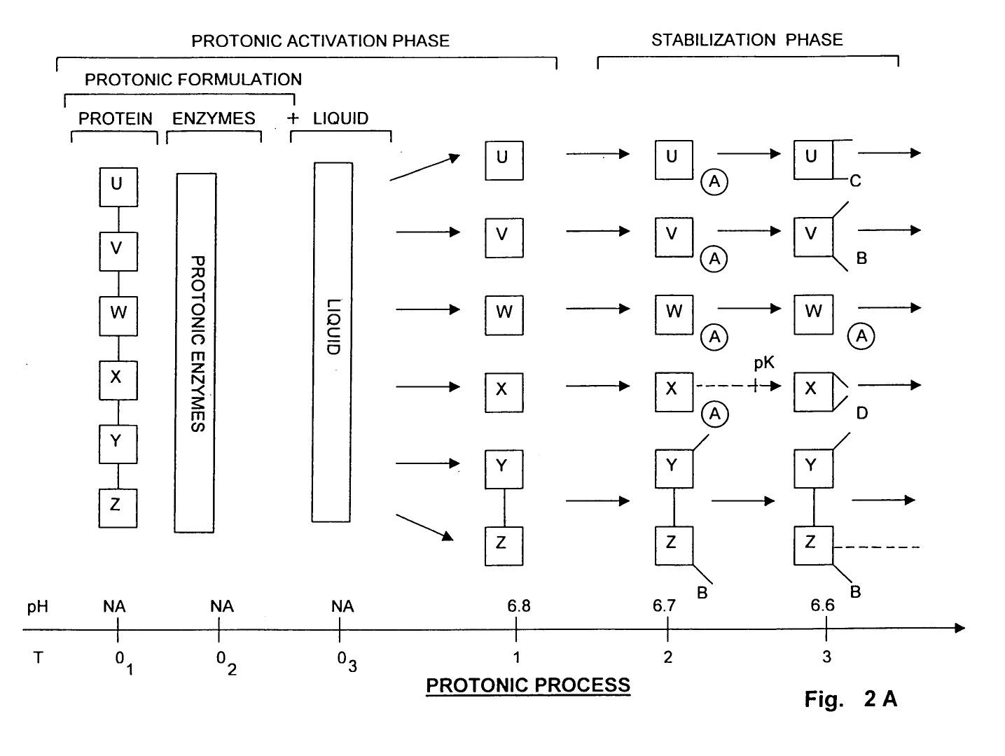

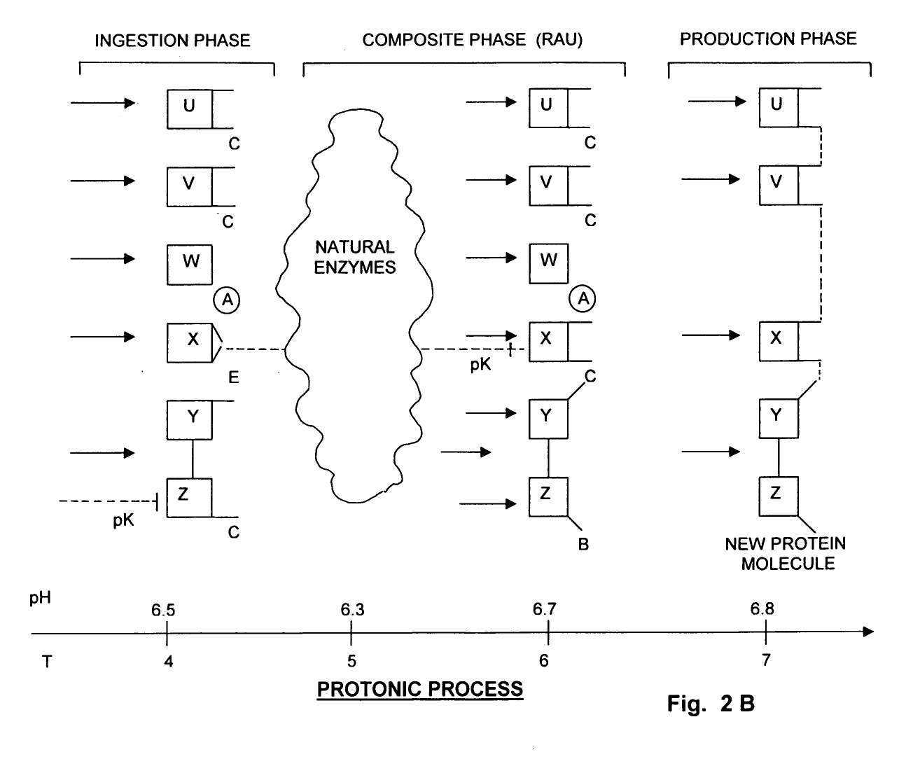

FIGS. 2A and 2B represent the protonic process in conjunction with a normal digestive process. Time lines 0₁, 0₂, and 0₃ represent the protein source in the protonic formulation, the enzymes in the protonic formulation, and constitution of the two by a liquid medium, respectively. It is not until the reconstitution of the protonic formulation by the liquid medium that the protonic activation process begins. Thereafter, at time line 1, the protein source u-v-w-x-y-z is broken down into four mono-peptides (u, v, w, and x) and one di-peptide (y-z). At time lines 2 and 3, the activation cycle begins and it is here that stabilization must be initiated. Stabilization does not inhibit continued activation or ‘de-activation’ (i.e., cycling from C to A or to E); but, does severely curtail such. With ingestion of the protonic mixture the body's natural digestive process also begins, further activation of non-activated amino acids may result, continued activation of pre-activated amino acids (from the protonic activation phase) may result (as illustrated by amino acid x), slight de-activation of pre-activated amino acids (from the protonic activation phase) may result (as illustrated by amino acid y-z). Note the pK for amino acid x is attained at a pH level which is slightly higher than pH 6.6 (see time lines 2 to 3) and the pK for amino acid y-z is at about pH level 6.5.

Clinical tests and studies have shown that, with use of the protonic mixture, about 30-40% more amino acids are utilized than when the protonic mixture is not used.

The present disclosure includes that contained in the present claims as well as that of the foregoing description. Although this invention has been described in its preferred forms with a certain degree of particularity, it is understood that the present disclosure of the preferred form has been made only by way of example and numerous changes in the details of formulation and combination and arrangement of elements and method steps may be resorted to without departing from the spirit and scope of the invention. Accordingly, the scope of the invention should be determined not by the embodiment[s] illustrated and described, but by the appended claims and their legal equivalents.

1. A protonic formulation (PF) comprising:
(a) a protein mixture (PM); and
(b) an enzyme-PH adjustor formulation (EAF) wherein said EAF comprises two or more of the following components selected from the group consisting of betaine HCl, pepsin, trypsin, chymotrypsin, protease, bromolein, papaya, vitamin C, lemon powder, glutamic acid, and glycine, wherein the proportion of said PM per gram to said EAF per gram is between about 10-30 grams of said PM to about each 1 gram of said EAF.

2. The formulation of claim 1 wherein the proportion of said PM per gram to said EAF per gram is about 25 grams said PM to about each 1 gram of said EAF.

3. The formulation of claim 1 wherein the percent by weight of said components of said EAF to a total weight of said EAF is
(a) for said betaine HCl between about 3.2% to about 4.8% of the total weight of said EAF,
(b) for said pepsin between about 1.2% to about 1.8% of the total weight of said EAF,
(c) for said trypsin between about 0.32% to about 0.48% of the total weight of said EAF,
(d) for chymotrypsin between about 0.24% to about 0.36% of the total weight of said EAF,
(e) for said protease between about 0.32% to about 0.48% of the total weight of said EAF,
(f) for said bromolein between about 0.4% to about 0.6% of the total weight of said EAF,
(g) for said papaya between about 0.48% to about 0.72% of the total weight of said EAF,
(h) for said vitamin c between about 4.0% to about 6.0% of the total weight of said EAF,
(i) for said lemon powder between about 0.48% to about 0.72% of the total weight of said EAF is
(j) for said glutamic acid between about 0.16% to about 0.24% of the total weight of said EAF, and
(k) for said glycine between about 69.12% to about 99.0% of the total weight of said EAF.

4. The formulation of claim 1 wherein the percent by weight of said components of said EAF to a total weight of said EAF,
(a) for said betaine HCl about 4.0% of the total weight of said EAF,
(b) for said pepsin about 1.5% of the total weight of said EAF,
(c) for said trypsin about 0.4% of the total weight of said EAF,
(d) for chymotrypsin about 0.3% of the total weight of said EAF,
(e) for said protease about 0.4% of the total weight of said EAF,
(f) for said bromolein about 0.5% of the total weight of said EAF,
(g) for said papaya about 0.6% of the total weight of said EAF,
(h) for said vitamin C about 5.0% of the total weight of said EAF,
(i) for said lemon powder about 0.6% of the total weight of said EAF,
(j) for said glutamic acid about 0.2% of the total weight of said EAF, and
(k) for said glycine about 86.4% of the total weight of said EAF.

5. The formulation of claim 1 further comprising a carrier for said PF.

6. The formulation of claim 5 wherein the proportion of said carrier by milliliter to said PF by gram is between about 120-240 milliliters of said carrier to about each 11-31 grams of said PF.

7. The formulation of claim 5 wherein the proportion of said carrier by milliliter to said PF by gram is about 180 milliliters of said carrier to about each 26 grams of said PF.

8. The formulation of claim 5 wherein said carrier is a liquid substance.

9. The formulation of claim 5 further comprising a stabilizer wherein the proportion of said stabilizer by milliliter to said PF by gram is between about 2-10 milliliters of said stabilizer to between about each 11-31 grams of said PF.

10. The formulation of claim 9 wherein the proportion of said said stabilizer by milliliter to said PF by gram is about 7.5 milliliters of said stabilizer to about each 26 grams of said PF.

11. The formulation of claim 1 wherein said PM comprises two or more of the following components selected from the group consisting of whey protein isolate, instant whey concentrate, soy protein isolate, pea protein, rice protein, maltodextrin, steviocide, french vanilla flavor, peach mango flavor, xanthan gum, lecithin, and tricalcium phosphate.

12. The formulation of claim 111 wherein the percent by weight of said components of said PM to a total weight of said PM is
(a) for said whey protein isolate between about 24.0% to about 36.0% of the total weight of said PM,
(b) for said instant whey concentrate between about 12.0% to about 18.0% of the total weight of said PM,
(c) for said soy protein isolate between about 20.0% to about 30.0% of the total weight of said PM,
(d) for said pea protein between about 4-0.0% to about 6.0% of the total weight of said PM,
(e) for said rice protein between about 4.0% to about 6.0% of the total weight of said PM,
(f) for said maltodextrin between about 12.5% to about 19.0% of the total weight of said PM,
(g) for said steviocide between about 0.24% to about 0.36% of the total weight of said PM,
(h) for said french vanilla flavor between about 1.4% to about 2.1% of the total weight of said PM,
(i) for said peach mango flavor between about 0.4% to about 0.6% of the total weight of said PM,
(j) for said xanthan gum between about 0.4% to about 0.6% of the total weight of said PM,
(k) for said lecithin between about 0.4% to about 0.6% of the total weight of said PM, and
(l) for said tricalcium phosphate between about 0.6% to about 0.9% of the total weight of said PM.

13. The formulation of claim 11 wherein the percent by weight of said components of said PM to a total weight of said PM is
(a) for said whey protein isolate about 30.0% of the total weight of said PM,
(b) for said instant whey concentrate about 15.0% of the total weight of said PM,
(c) for said soy protein isolate about 25.0% of the total weight of said PM,
(d) for said pea protein about 5.0% of the total weight of said PM,
(e) for said rice protein about 5.0% of the total weight of said PM,
(f) for said maltodextrin about 15.7% of the total weight of said PM,
(g) for said steviocide about 0.3% of the total weight of said PM,
(h) for said french vanilla flavor about 1.75% of the total weight of said PM,
(i) for said peach mango flavor about 0.5% of the total weight of said PM,
(j) for said xanthan gum about 0.5% of the total weight of said PM,
(k) for said lecithin about 0.5% of the total weight of said PM, and
(l) for said tricalcium phosphate about 0.75% of the total weight of said PM.

14. A protonic formulation (PF) comprising:
(a) a protein mixture (PM), wherein said PM comprises two or more of the following components selected from the group consisting of whey protein isolate, instant whey concentrate, soy protein isolate, pea protein, rice protein, maltodextrin, steviocide, french vanilla flavor, peach mango flavor, xanthan gum, lecithin, and tricalcium phosphate; and
(b) an enzyme-PH adjustor formulation (EAF), wherein the proportion of said PM per gram to said EAF per gram is between about 10-30 grams of said PM to about each 1 gram of said EAF.

15. The formulation of claim 14 wherein the percent by weight of said components of said PM to a total weight of said PM is
(a) for said whey protein isolate between about 24.0% to about 36.0% of the total weight of said PM,
(b) for said instant whey concentrate between about 12.0% to about 18.0% of the total weight of said PM,
(c) for said soy protein isolate between about 20.0% to about 30.0% of the total weight of said PM,
(d) for said pea protein between about 4.0% to about 6.0% of the total weight of said PM,
(e) for said rice protein between about 4.0% to about 6.0% of the total weight of said PM,
(f) for said maltodextrin between about 12.5% to about 19.0% of the total weight of said PM,
(g) for said steviocide between about 0.24% to about 0.36% of the total weight of said PM,
(h) for said french vanilla flavor between about 1.4% to about 2.1% of the total weight of said PM,
(i) for said peach mango flavor between about 0.4% to about 0.6% of the total weight of said PM,
(j) for said xanthan gum between about 0.4% to about 0.6% of the total weight of said PM,
(k) for said lecithin between about 0.4% to about 0.6% of the total weight of said PM, and
(l) for said tricalcium phosphate between about 0.6% to about 0.9% of the total weight of said PM.

16. The formulation of claim 14 wherein the percent by weight of said components of said PM to a total weight of said PM is
(a) for said whey protein isolate about 30.0% of the total weight of said PM,
(b) for said instant whey concentrate about 15.0% of the total weight of said PM,
(c) for said soy protein isolate about 25.0% of the total weight of said PM,
(d) for said pea protein about 5.0% of the total weight of said PM,
(e) for said rice protein about 5.0% of the total weight of said PM,
(f) for said maltodextrin about 15.7% of the total weight of said PM,
(g) for said steviocide about 0.3% of the total weight of said PM,
(h) for said french vanilla flavor about 1.75% of the total weight of said PM,
(i) for said peach mango flavor about 0.5% of the total weight of said PM,
(j) for said xanthan gum about 0.5% of the total weight of said PM,
(k) for said lecithin about 0.5% of the total weight of said PM, and
(l) for said tricalcium phosphate about 0.75% of the total weight of said PM.

17. The formulation of claim 14 wherein the proportion of said PM per gram to said EAF per gram is about 25 grams said PM to about each 1 gram of said EAF.

18. The formulation of claim 14 further comprising a carrier for said PF.

19. The formulation of claim 18 wherein the proportion of said said carrier by milliliter to said PF by gram is between about 120-240 milliliters of said carrier to about each 11-31 grams of said PF.

20. The formulation of claim 18 wherein the proportion of said carrier by milliliter to said PF by gram is about 180 milliliters of said carrier to about each 26 grams of said PF.

21. The formulation of claim 18 wherein said carrier is a liquid substance.

22. The formulation of claim 18 further comprising a stabilizer wherein the proportion of said stabilizer by milliliter to said PF by gram is between about 2-10 milliliters of said stabilizer to between about each 11-31 grams of said PF.

23. The formulation of claim 22 wherein the proportion of said stabilizer by milliliter to said PF by gram is about 7.5 milliliters of said stabilizer to about each 26 grams of said PF.

24. The formulation of claim 14 wherein said EAF comprises two or more of the following components selected from the group consisting of betaine HCl, pepsin, trypsin, chymotrypsin, protease, bromolein, papaya, vitamin C, lemon powder, glutamic acid, and glycine.

25. The formulation of claim 24 wherein the percent by weight of said components of said EAF to a total weight of said EAF,
(a) for said betaine HCl is between about 3.2% to about 4.8% of the total weight of said EAF,
(b) for said pepsin is between about 1.2% to about 1.8% of the total weight of said EAF,
(c) for said trypsin is between about 0.32% to about 0.48% of the total weight of said EAF,
(d) for chymotrypsin is between about 0.24% to about 0.36% of the total weight of said EAF,
(e) for said protease is between about 0.32% to about 0.48% of the total weight of said EAF,
(f) for said bromolein is between about 0.4% to about 0.6% of the total weight of said EAF,
(g) for said papaya is between about 0.48% to about 0.72% of the total weight of said EAF,
(h) for said vitamin C is between about 4.0% to about 6.0% of the total weight of said EAF,
(i) for said lemon powder is between about 0.48% to about 0.72% of the total weight of said EAF,
(j) for said glutamic acid is between about 0.16% to about 0.24% of the total weight of said EAF, and
(k) for said glycine is between about 69.12% to about 99.0% of the total weight of said EAF.

26. The formulation of claim 24 wherein the percent by weight of said components of said EAF to a total weight of said EAF,
(a) for said betaine HCl is about 4.0% of the total weight of said EAF,
(b) for said pepsin is about 1.5% of the total weight of said EAF,
(c) for said trypsin is about 0.4% of the total weight of said EAF,
(d) for chymotrypsin is about 0.3% of the total weight of said EAF,
(e) for said protease is about 0.4% of the total weight of said EAF,
(f) for said bromolein is about 0.5% of the total weight of said EAF,
(g) for said papaya is about 0.6% of the total weight of said EAF,
(h) for said vitamin C is about 5.0% of the total weight of said EAF,
(i) for said lemon powder is about 0.6% of the total weight of said EAF,
(j) for said glutamic acid is about 0.2% of the total weight of said EAF, and
(k) for said glycine is about 86.4% of the total weight of said EAF.
